(19) United States
(12) Patent Application Publication
(10) Pub. No.: US 2006/0286657 A1
(43) Pub. Date: Dec. 21, 2006

(54) Novel bioconjugation reactions for acylating polyethylene glycol reagents

(76) Inventors: Samuel Zalipsky, Redwood City, CA (US); Radwan Kiwan, Albany, CA (US)

Correspondence Address: PERKINS COIE LLP, P.O. BOX 2168, MENLO PARK, CA, 94026, US

(21) Appl. No.: 11/445,833

(22) Filed: Jun. 1, 2006

Related U.S. Application Data
(60) Provisional application No. 60/686,738, filed on Jun. 1, 2005.

(51) Int. Cl. C12N 9/36; C07K 14/505; C07K 14/475

(52) U.S. Cl. 435/206; 530/391.1; 530/399; 525/54.1

(57) ABSTRACT

A method for conjugation of PEG to a protein in an aqueous solution at a pH less than or equal to about 7.0 or neutral pH comprising combining an activated PEG reagent and a protein in the presence of an activating agent at a pH of less than about 7.0 or neutral pH. In one embodiment, the method produces mixed populations of moderately PEGylated proteins, including 1:1, 2:1, and 3:1 PEGylated proteins.

This application claims the benefit of U.S. Provisional Patent Application Ser. No. 60/686,738, filed Jun. 1, 2005, which is incorporated herein by reference.

### BACKGROUND

Due to recent advances in genetic and cell engineering technologies, peptides and proteins known to exhibit various pharmacological actions in vivo can be produced in quantities useful for pharmaceutical applications. A limitation to the development of these therapeutics is the preparation of stable pharmaceutical compositions of the proteins.

Therapeutic proteins are typically administered by frequent injection over time because the proteins often have a short in vivo half-life and negligible oral bioavailability. In some cases, frequent injection regimens pose a physical burden on the patient and are associated with increased administrative costs, as compared to costs associated with administering small molecules. As such, there is currently a great deal of interest in developing and evaluating longer lasting or sustained-release compositions and formulations.

Effective sustained-release compositions and formulations can provide a means of controlling blood levels of the active ingredient, and also provide greater efficacy, safety, patient convenience and patient compliance.

To date, two of the most widely used approaches to obtain sustained-action of a protein therapeutic include: 1) modifying the protein to increase the circulating half-life of the protein, for example by increasing the molecular weight and reducing immunogenicity; and 2) encapsulating the protein, for example, in polymer microspheres.

In connection with the first mechanism, i.e., protein modification, conjugation of biologically active molecules with biocompatible polymers is one way to improve formulation properties and in vivo performance of such molecules. Polyethylene glycol (PEG) is one of the most useful polymers often employed for this purpose. The properties that are attainable by PEG attachment to various low and high molecular weight drugs, and the corresponding applications of the resulting macromolecular conjugates have been extensively documented (for a review, see Zalipsky, Bioconjugate Chem., 6:150-165, 1995 and Adv. Drug Delivery Rev., 16:157-182, 1995). In particular, proteins are often conjugated with PEG, usually methoxy-PEG (mPEG), to gain longer in vivo circulation, reduced immunogenicity, and improved solubility and resistance to proteolytic enzymes.

PCT publication WO 02/049673 (Burg et al.) “refers to conjugates of erythropoietin with poly(ethylene glycol) comprising an erythropoietin glycoprotein having the in vivo biological activity of causing bone marrow cells to increase production of reticulocytes and red blood cells and selected from the group consisting of human erythropoietin and analogs thereof which have sequence of human erythropoietin modified by the addition of from 1 to 6 glycosylation sites or a rearrangement of at least one glycosylation site; said glycoprotein being covalently linked to one poly(ethylene glycol) group of the formula —CO—(CH₂)ₓ)—(OCH₂CH₂)ₘ—OR with the —CO of the poly(ethylene glycol) group forming an amide bond with amino groups; wherein R is lower alkyl; x is 2 or 3; and m is from about 450 to about 1350.”

PCT publication WO 02/32957 (Nakamura et al.) describes PEG-modified EPO prepared by “chemically modifying the lysine residue at the 52-position of natural erythropoietin (natural EPO) with polyethylene glycol,” which PEG-modified EPO is stated to show a “ . . . long-lasting drug effect.”

PCT publication WO 97/24440 (De Sauvage et al.) describes “OB protein-immunoglobulin chimeras and polyethylene glycol (PEG)-OB derivatives” stated to have “extended half-life as compared to the corresponding native OB proteins.”

PCT publication WO 94/28024 (Chyi et al.) describes “[b]iologically active conjugates of glycoproteins having erythropoietic activity and having at least one oxidized carbohydrate moiety covalently linked to a non-antigenic polymer.”

PCT publication WO 90/13540 (S. Zalipsky) describes poly(ethylene glycol)-N-succinimide carbonate and its preparation.

Many methods are available for linking mPEG to proteins, usually to their amino groups of lysine residues or N-terminal of the polypeptide sequence (Zalipsky & Lee, 1992, supra, “Use of Functionalized Polyethylene Glycols for Modification of Polypeptides,” in Poly(Ethylene Glycol) Chemistry: Biotechnical and Biomedical Applications, J. M. Harris, ed., Plenum, New York N.Y., pp. 347-370). Urethane (carbamate) attachment of PEG to a protein is a convenient way to form PEG-protein conjugates. Carbamate linkages are more resistant to hydrolysis than amide linkages, which are also often utilized for protein PEGylation. Thus the urethane-linked conjugates are very stable in a variety of physiological conditions. There are a few known PEG reagents that are used to make urethane linked PEG-proteins (Zalipsky & Lee, 1992; Veronese et al., 1985, Appl. Biochem. Biotechnol., 11:141-152). These include slow-reacting imidazolyl formate, trichlorophenyl carbonate, and nitrophenyl carbonate (NPC) derivatives. A more reactive reagent, mPEG-succinimidyl carbonate (mPEG-SC), is often utilized (e.g., U.S. Pat. Nos. 5,122,614, 5,324,844, 5,612,460 and 5,808,096 to Zalipsky). As a rule, in comparison to less reactive reagents, a more reactive reagent allows faster, more efficient reaction under milder conditions. On the other hand, the less reactive reagents have better storage stability, and usually better selectivity.

PEGylation of proteins with slow-reacting reagents such as PEG-NPC proceeds more efficiently at a pH range of 8-10, as most amino groups of proteins are deprotonated and are highly reactive in this pH range. Many proteins are either not soluble or not stable at this basic pH range. Under these conditions, multiple amino groups react randomly with low selectivity. On the other hand, PEG-NPC is not very reactive at pH<8, thus these reactions proceed very slowly and not efficiently. This reagent is essentially unreactive at neutral and acidic pH (about 5-7).

The foregoing examples of the related art and limitations related therewith are intended to be illustrative and not exclusive. Other limitations of the related art will become apparent to those of skill in the art upon a reading of the specification and upon study of the drawings.

### SUMMARY

The following aspects and embodiments thereof described and illustrated below are meant to be exemplary and illustrative, not limiting in scope.

In one aspect, an alternative method of generating PEGylated peptides and proteins is provided. In a preferred embodiment, the method allows for efficient modification of proteins at a pH at or below about 7 or about 6. The method allows for efficient formation of urethane-linked PEG proteins using mild PEG reagents and mild reaction conditions. The process results in the formation of moderately PEGylated proteins. In one embodiment, the method is advantageous for the modification of proteins that are not stable or not soluble at or above a neutral pH range. In another embodiment, the method is useful to PEGylate a protein that is insoluble or unstable at a pH higher than about 7.0 or 8.0.

In another aspect, a method useful for optimizing the yield of 1:1 PEG-protein in a heterogeneous population of PEG-protein molecules is described. This method comprises combining a PEG derivatized acylating agent and a protein in the presence of an activating agent at a pH of less than about 7.0 or neutral pH. In embodiments, the PEG derivatized acylating agent is mPEG-NPC. In another embodiment, the activating agent is selected from the group consisting of HOSu, HOBt, and HOAt.

In yet another aspect, the method is useful to maximize the yield of minimally conjugated proteins, i.e. 1:1, 1:2, and 1:3 protein:PEG.

In a further aspect, a method useful for PEGylating a protein that is insoluble or unstable at a pH higher than about 7.0 or neutral pH is described. The method comprises reacting the protein with a PEG derivatized acylating agent and an activating agent at a pH lower than about 7.0. In embodiments, The PEG derivatized acylating agent is mPEG-NPC. In another embodiment, the activating agent is selected from the group consisting of HOSu, HOBt, and HOAt.

In an additional aspect, a method to PEGylate a protein to form predominately 1:1 PEG-protein at a pH of less than about 8 and greater than the pKa of the additive to boost the rate of the reaction is described. The method comprises reacting the protein with a PEG derivatized acylating agent and an activating agent at a pH lower than about 8.0. In an embodiment, The PEG derivatized acylating agent is mPEG-NPC. In another embodiment, the activating agent is selected from the group consisting of HOSu, HOBt, and HOAt.

In addition to the exemplary aspects and embodiments described above, further aspects and embodiments will become apparent by reference to the drawings and by study of the following descriptions.

### BRIEF DESCRIPTION OF THE DRAWINGS

FIG. 1 illustrates a reaction scheme for PEGylation of proteins using mPEG-NPC at neutral pH;

FIG. 2 is a trace of HPLC-SEC analysis of PEG30k-EPO conjugates and free EPO plotted as mVolts over time in minutes;

FIGS. 3A-3D are graphs of the separation with an ion exchange column (FIG. 3A) of the conjugation reaction sample plotted as mAU over time in minutes; and the fractions from the ion exchange column analyzed by HPLC-SEC (FIG. 3B-3D) plotted as Volts or mVolts over time in minutes;

FIG. 4 is a graph of HPLC-SEC analysis of a protein determination assay for mPEG30k-EPO plotted as mVolts over time in minutes;

FIGS. 5A-5B show SDS-PAGE for the PEG30k-EPO conjugates with iodine stain (FIG. 5A) and Coomassie blue stain (FIG. 5B);

FIGS. 6A-6B are graphs of HPLC-SEC analysis of PEG30k-EPO conjugates formed at pH 6.5, 7.0, or 8.0, without (FIG. 6A) and with (FIG. 6B) an added activating agent (HOSu) in the buffer plotted as mVolts over time in minutes;

FIGS. 7A-7B are graphs of the OD of p-nitrophenol after hydrolysis of mPEG30k-NPC in MOPS and MOPS/HOSu buffers, respectively, at 400 nm over time in minutes;

FIG. 8 is an SDS-PAGE of the PEG30k-EPO conjugates formed at various molar ratios of PEG:EPO at pH 7, where lane 1 is free EPO; lane 2 is PEG:EPO at a molar ratio of 3:1 in the presence of HOSu; lane 3 is PEG:EPO at a molar ratio of 6:1 in the presence of HOSu; lane 4 is PEG:EPO at a molar ratio of 9:1 in the presence of HOSu; lane 5 is PEG:EPO at a molar ratio of 3:1 in the presence of HOBt; lane 6 is PEG:EPO at a molar ratio of 6:1 in the presence of HOBt; lane 7 is PEG:EPO at a molar ratio of 9:1 in the presence of HOBt; lane 8 is PEG:EPO at a molar ratio of 6:1 with no activating agent; lane 9 is the molecular weight standard; lane 10 is 1 mg/ml PEG:EPO at a molar ratio of 3:1 in the presence of HOSu; lane 11 is 2 mg/ml PEG:EPO at a molar ratio of 3:1 in the presence of HOSu; lane 12 is PEG blocked with glycine;

FIG. 9 is a graph of HPLC-SEC for PEG30k-BMP7 conjugates and a BMP7 reference plotted as mVolts over time in minutes;

FIG. 10A is a graph of separation of PEG30k-BMP7 by cation exchange chromatography plotted as mAU over time in minutes. FIGS. 10B-10C illustrate the fractions from the cation exchange compared to the BMP7 control and the conjugation reactions as analyzed by HPLC-SEC plotted as mVolts over time in minutes;

FIG. 11 is a graph of the fluorescence intensity for purified PEG30k-BMP7 conjugates plotted as mVolts over time in minutes; and

FIGS. 12A-12B show electrophoresis gels for the PEG30k-BMP7 conjugates with iodine stain (FIG. 12B) and Coomassie blue stain (FIG. 12A).

### DETAILED DESCRIPTION

I. Definitions

The terms below have the following meanings unless indicated otherwise.

“Peptide” as used herein refers to any of the various amides that are derived from two or more α-amino acids by combination of the amino group of one acid with the carboxyl group of another. Peptides may be obtained by partial hydrolysis of proteins. “Polypeptide” as used herein refers to a chain of peptides. “Protein” as used herein refers to any of the numerous naturally occurring, usually extremely complex, substances that consist of amino-acid residues joined by peptide bonds. Proteins may further contain carbon, hydrogen, nitrogen, oxygen, usually sulfur, and occasionally other elements (such as phosphorus or iron). Proteins are generally characterized by a biological function including, for example, enzymes, hormones, or immunoglobulins. Unless specifically stated or recognizable by context, these terms are used interchangeably herein.

“Hydrophilic polymer” as used herein refers to a polymer having moieties soluble in water, which lend to the polymer some degree of water solubility at room temperature. Exemplary hydrophilic polymers include polyvinylpyrrolidone, polyvinylmethylether, polymethyloxazoline, polyethyloxazoline, polyhydroxypropylyoxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Properties and reactions with many of these polymers are described in U.S. Pat. Nos. 5,395,619 and 5,631,018.

“PEGylation” refers to the attachment of one or more polyethylene glycol (PEG) substituent or derivatives to a biologically active protein.

Acylating agent” refers to an agent capable of connecting an acyl group to another chemical compound, whereby the acylating agent provides the acyl group. Exemplary acylating reagents include nitrophenyl carbonate, trichlorophenyl carbonate, pentachlorophenyl carbonate, and carbonyl imidazole as well as various active esters, e.g. nitrophenyl ester, pentafluoroethyl ester, trichlorophenyl ester.

“PEG derivatized acylating reagent” refers to an acylating agent that is derivatized to include polyethylene glycol.

Abbreviations: PEG: polyethylene glycol; mPEG: methoxy polyethylene glycol; HOSu: N-hydroxysuccinimide; HOBt: N-Hydroxybenzotriazole; NPC: nitrophenyl carbonate; DTB: dithiobenzyl; SC: succinimidyl carbonate.

II. Method of Forming Conjugates

The solubility of proteins in aqueous solutions varies enormously based on the protein structure and composition. While some proteins are very soluble, many proteins, especially structural proteins are essentially insoluble under physiological conditions and exist normally as solids. (Creighton, Proteins: Structures and Molecular Properties, W.H. Freeman and Company, N.Y. (1993)). The solubility of proteins is typically lowest near pl≈pH.

As noted above, however, PEGylation of proteins with typical acylating reagents such as PEG-NPC proceeds quickly at a pH greater than 7.5, typically greater than 8, as PEG-NPC is highly reactive in this pH range, but is not very reactive at neutral or lower pH values where the reaction proceeds very slowly and is not efficient.

The present methods take advantage of some of the benefits of low- and high-reactivity PEG reagents. Specifically, less reactive reagents, such as mPEG-NPC, have superior shelf life and selectivity characteristics as compared with more reactive reagents, yet their sluggish reactivity, particularly under neutral conditions, makes them undesirable for use. In contrast, the more reactive mPEG-SC and similar compounds suffer the disadvantages of being less stable and less selective than their less reactive counterparts, and can undergo undesirable side reactions (Zalipsky, Chem Com, 1:69-70, 1998).

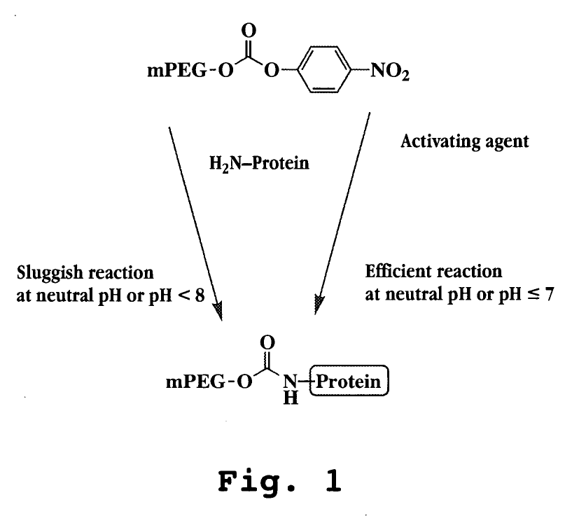

As seen in FIG. 1, use of an activating agent results in an efficient PEGylation reaction using an acylating derivative such as mPEG-NPC at neutral pH or a pH≦7. Thus, the method of using an acylating derivative as a reagent for formation of amide- or urethane-linked PEG proteins is modified for use under conditions that increase the reaction efficiency and allow facile protein modification under neutral pH or below pH 7.0 conditions. In a preferred embodiment, the acylating derivative is a PEG derivatized acylating reagent. Although, the method is described with reference to PEG derivatized acylating reagents, it will be appreciated that other hydrophilic polymer derivatized acylating reagents are suitable for use with the method described herein.

It will be appreciated that many water-soluble, non-carboxylic, Brönstead acids of moderate acidity having the propensity to donate N- or O-linked protons to the PEGylation reagent are suitable for use as the activating agent in the present methods. General examples include acidic alcohols, phenols, imidazols, triazols and tetrazols, among others. Examples of acidic acids suitable for use in this aspect of the invention include, but are not limited to, N-hydroxydicarboxyimides, N-hydroxyphthalimides particularly with nitro and other electron withdrawing substituents on the aromatic ring, N-hydroxy tetrahydrophthalimide, N-hydroxyglutarimide, N-hydroxy-5-norbornene-2,3-dicarboxyimide, and N-hydroxy-7-oxabicyclo[2.21]hept-5-ene-2,3-dicarboxyimide. 1-N-hydroxybenzotriazol and derivatives with electron withdrawing groups on the aromatic ring, e.g. nitro, chloro, 3-hydroxy-1,2,3-benzotriazin-4(3H)-one. N-hydroxysulfosuccinimide sodium salt is very soluble in water, which means that it can be used at even higher concentration in aqueous buffers than HOSu. Exemplary hydroxy amine derivatives include N-hydroxysuccinimide (HOSu), sulfonate derivatives of HOSu, 1-hydroxybenzotriazole (HOBt), and hydroxyl-7-azabenzotriazole (HOAt). These coupling agents can act as an efficient buffer component in a pH range of about 4 to about 7.5, based on their pKa. For example, HOSu, being a weak acid of pKa=6, acts as an efficient buffer component in a pH range of about 5 to about 7. The coupling reagent may be added to a buffer, or may comprise the buffer with or without other salts. Further, as HOSu is quite soluble in an aqueous solution, it can be added to buffers at relatively high concentration to further boost the PEGylation reaction.

Exemplary phenols include, but are not limited to, dinitrophenol, trinitrophenol, trifluorophenol, pentafluorophenol, and pentachlorophenol. It should be noted that water solubility is a factor for pentafluorophenol and pentachlorophenol. In addition, 4- or 2-hydroxypyridine and derivatives are also suitable for use in the present methods as exemplified by hydroxyl-2-nitropyridine.

Other exemplary compounds for use as the activating agent include compounds having an acidic N—H functionality, such as imidazol derivatives with electron withdrawing groups (imidazol, pKa=7), e.g. 4- or 2-nitroimidazol, triazol, tetrazol, and some derivatives, such as 2-nitro-1,2,4-triazole.

As mentioned above, in one exemplary embodiment, the invention relates to a method using acylating PEG reagents of low to medium reactivity, particularly reagents having low to medium reactivity at room temperature and/or at pH≦7.0. A particularly preferred acylating reagent is mPEG-NPC. Nitrophenyl carbonate derivatized polyethylene glycol is exemplified for use in this aspect of the present invention, however, as seen in Example 4, the method has also been applied to preparation of thiolytically cleavable dithiobenzyl (DTB) urethane-linked PEG-protein conjugates, utilizing a mPEG-DTB-NPC reagent. The reactivity and selectivity benefits were similar to those obtained using the non-cleavable mPEG-NPC reagent. Other low-reactivity PEG reagents suitable for use in the present invention include, but are not limited to, carbonyl imidazolyls, trichlorophenyl carbonates, and other nitrophenyl carbonates, which have been utilized to make urethane-linked PEG-proteins (see, e.g. Zalipsky & Lee, 1992, supra). Likewise, methoxy-PEG (mPEG) is exemplified herein, but is not the only modified PEG that can be used. Other modified PEGs, preferably rendered monofunctional by addition of an inert group to one end, are also suitable for use in the method. Examples include, but are not limited to, short alkoxy PEG derivatives (ethoxy, butoxy, and the like) or PEG modified with various protected functional groups, as would be understood by one of skill in the art.

It will further be appreciated that while a PEG reagent comprising 12,000 and 30,000 Da PEG is exemplified herein, other PEG lengths are also contemplated for use in the present method. A preferred size range of PEG is 1,000-50,000 Da. Preferably, the PEG length is 40,000 Da or less, more preferably 30,000 Da or less.

In one embodiment, the method preferably includes the step of combining the protein with an activating agent and mPEG-NPC in an aqueous medium at a pH of less than about 8.0.

In one preferred embodiment, the pH is about 5.0 or 6.0 to about 7.5. In other embodiments, the pH is less than about 7 or 8. It will be appreciated that neutral conditions are often more favorable for protein stability and/or solubility. Neutral conditions also generally favor modification of the most reactive and least basic amino groups on a protein. As the N-terminal amino group is usually a few orders of magnitude less basic (pKa=7.9) than the ε-amino group of lysine (pKa=10-11), lower pH tends to keep most of the latter groups fully protonated. Therefore, a lower pH is generally more favorable for selective modification of the N-terminal amino group. It will be appreciated that often proteins PEGylated predominantly on the N-terminal amino retain higher functional activity.

The protein, activating agent, and mPEG-NPC are combined for a period of time from about 0.5 hours to about 24 hours. In one embodiment, the time is from about 2 hours to about 6 hours. It will be appreciated that one of skill in the art can readily determine and/or vary the time to optimize the reaction.

The reaction temperature is generally about room temperature, or between 8° C. and 37° C., however, it will be appreciated that one of skill in the art can readily determine and/or vary the temperature to optimize the reaction.

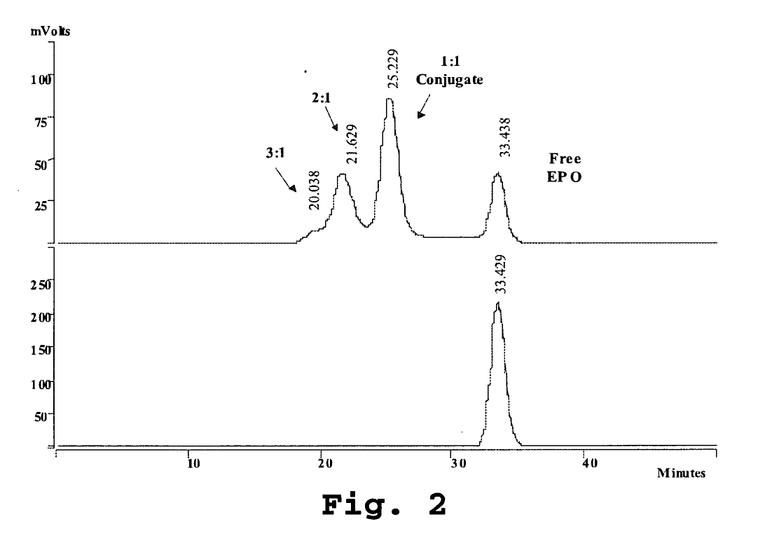

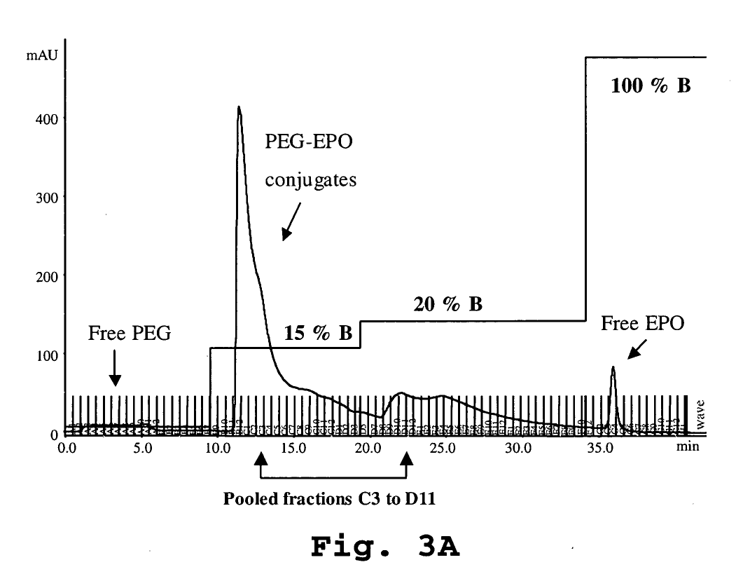

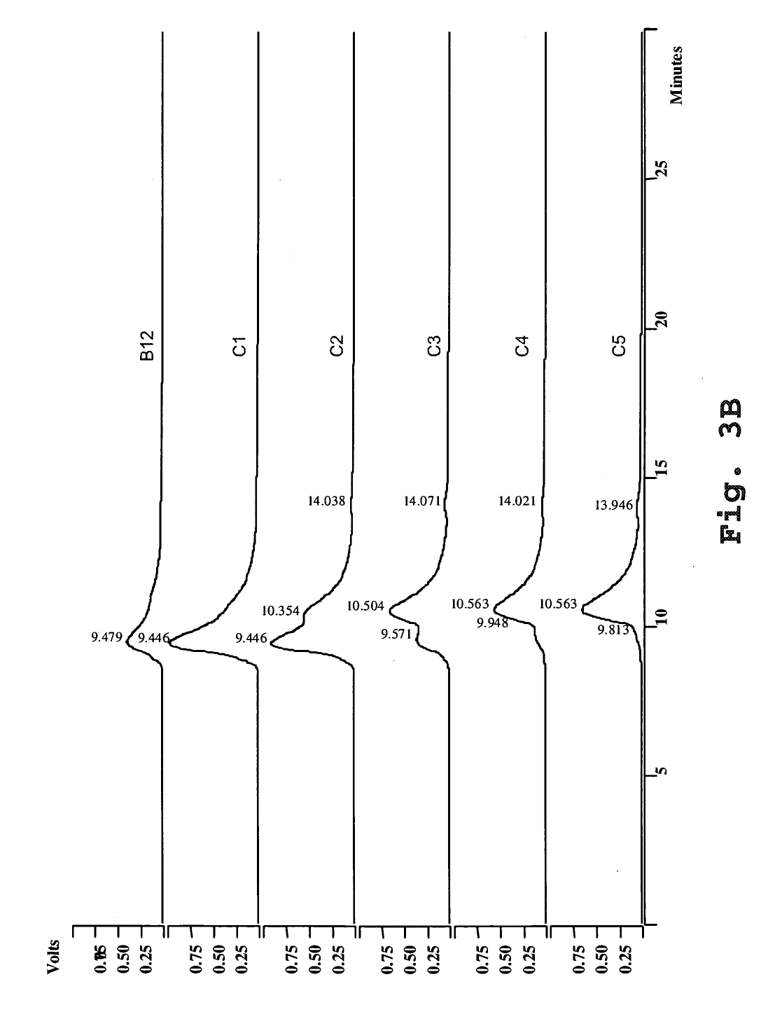

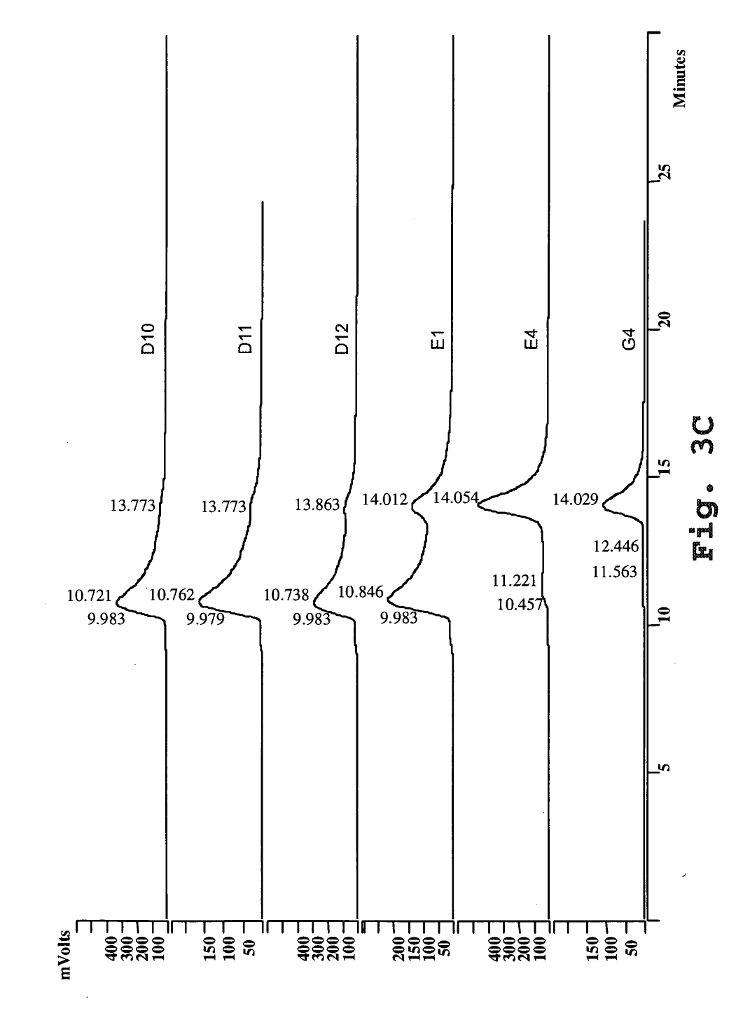

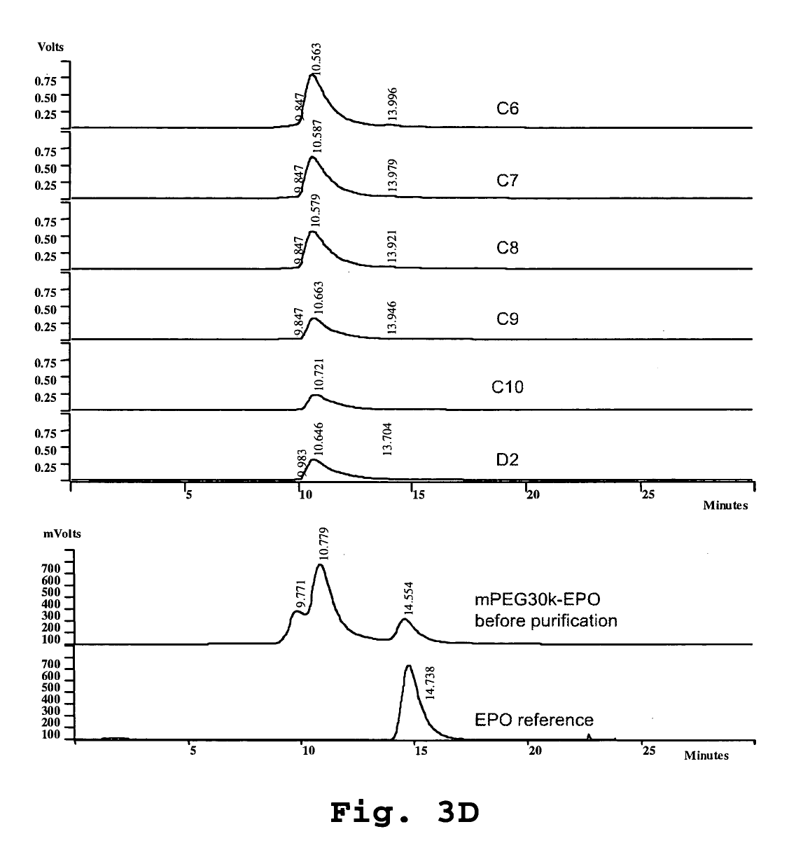

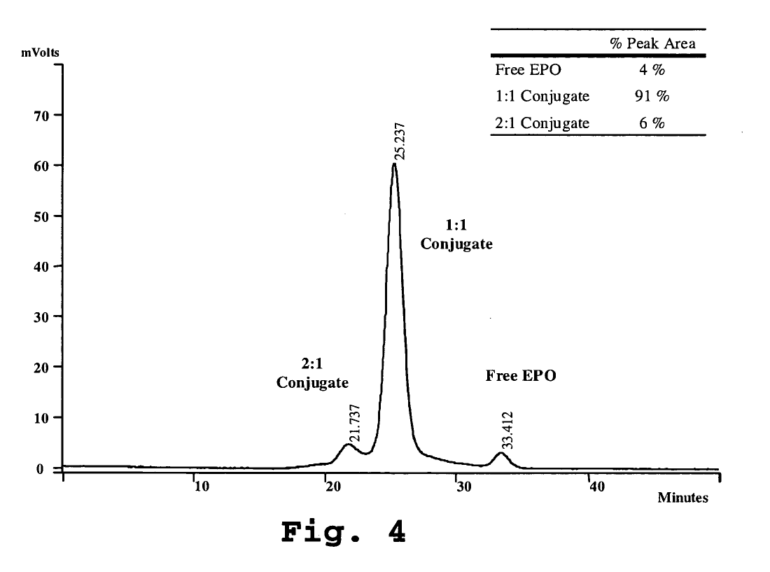

As described in Example 1, EPO was conjugated to mPEG30k-NPC using the HOSu in the in situ activation process. The reaction was efficient with 81% of the EPO being conjugated, leading to the formation of mainly mono- and di-PEGylated-EPO as seen in FIG. 2. FIG. 2 shows a trace of the HPLC-SEC analysis of the PEG30k-EPO conjugates formed in Example 1 (top panel) as compared to free EPO (bottom panel). Free EPO eluted at about 33 minutes, where the conjugates eluted at about 25 minutes for the 1:1 conjugate, at about 21 minutes for the 2:1 conjugate, and at about 20 minutes for the 3:1 conjugate. The peak area for each of the conjugates and the free EPO was calculated and is shown in the figure. Specifically, 78% of the EPO was conjugated as mono-(50%) and di-PEGylated-EPO (28%). As seen in FIG. 3A, the ion exchange purification was able to separate unreacted EPO and PEG from the PEGylated-EPO. Because the aim of this preparation was to produce a lightly PEGylated conjugate containing a majority of mono-PEG-EPO, a few fractions from the ion exchange purification containing 2:1 and 3:1 conjugates were not included in the final purified sample. The total amount of mPEG30k-EPO prepared was approximately 4.3 mg, containing over 90% of mono-PEGylated protein. FIGS. 3B-3D depict the results of HPLC-SEC analysis of the B12, C1-C10, D2, D10-D12, E1, E4, and G4 fractions. As seen in FIGS. 3B and 3C, the majority of the fractions show a significant amount of conjugation at about 9.5-10.5 minutes. As seen in FIG. 3D, the mPEG30k-EPO before purification showed significant conjugation at 9.771 and 10.779 minutes corresponding to the 2:1 and 1:1 conjugates, respectively. Thus, a majority of the fractions were predominantly 2:1 and 1:1 conjugates. As further seen in FIG. 3D, the EPO reference eluted at about 14.738 minutes. FIG. 4 shows a trace of the HPLC-SEC analysis of the pooled C3 to D11 fractions of PEG30k-EPO conjugates as compared to free EPO. The free EPO eluted at about 33 minutes, while the 1:1 conjugates eluted at about 25 minutes and the 2:1 conjugates eluted at about 22 minutes. The pooled fractions gave the following composition: 91% 1:1 PEG-EPO, 6% 2:1 PEG-EPO and 4% free EPO as measured from the % peak area.

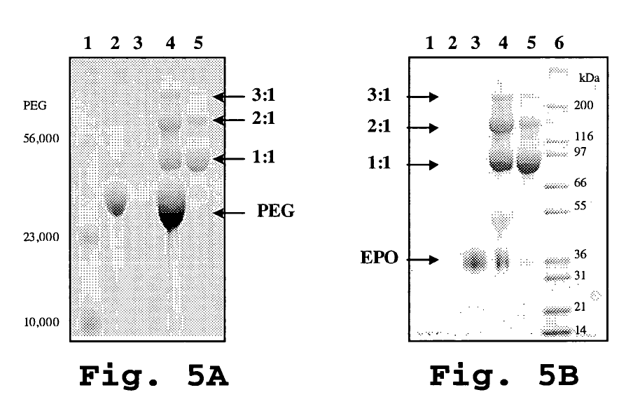

Electrophoresis gels were run and are shown in FIGS. 5A-5B. FIG. 5A shows a gel with an iodine stain for detection of the PEG. FIG. 5B shows a gel with a Coomassie blue stain for detection of protein. Lane 1 is the PEG molecular weight marker with PEG at 56,000 Da, 23,000 Da, and 10,000 Da, lane 2 is a PEG30k control, lane 3 is the EPO control, lane 4 is mPEG30k-EPO sample before purification. Lane 5 is the purified mPEG30k-EPO, and lane 6 is protein molecular weight markers (FIG. 5B only). FIG. 5A shows an excess of PEG in the reactions as seen by the band at about 30 Da. Lanes 4 and 5 show a majority of the conjugates were 1:1 and 2:1 conjugates as seen from the bands at about 95 kDa and about 150 kDa, respectively. As seen in lane 4, a number of 3:1 conjugates were also formed. As seen from these results, a majority of the conjugates are 1:1 conjugates.

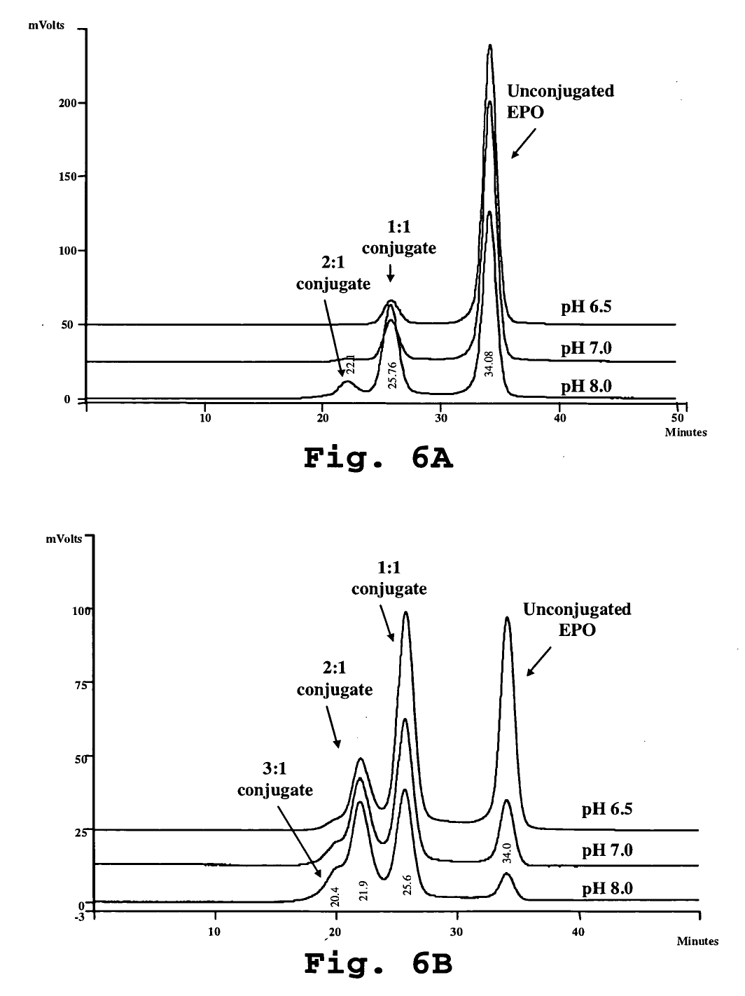

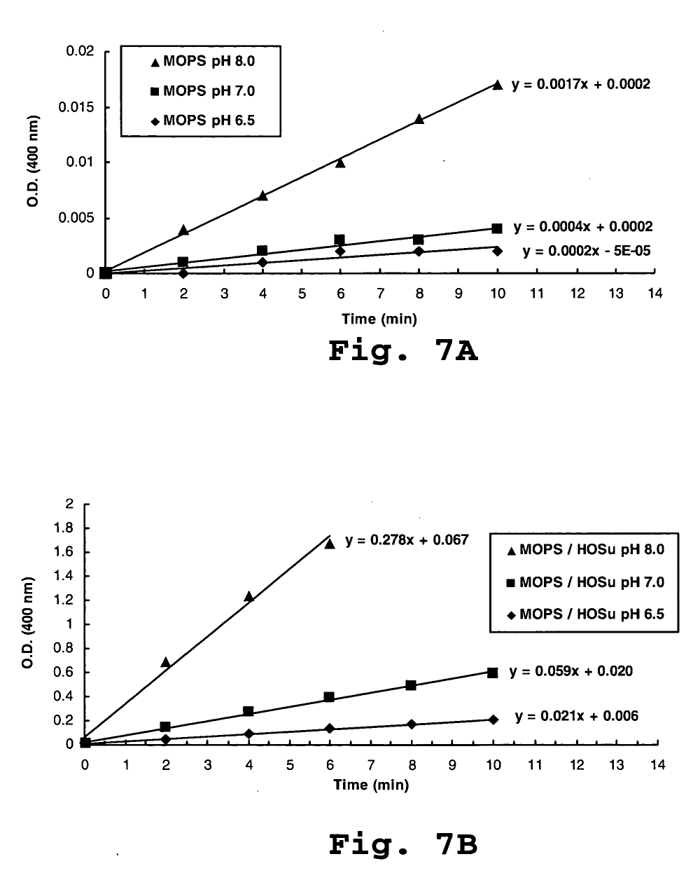

As detailed in Example 2, conjugates of mPEG30k-EPO were prepared in either MOPS buffer or in MOPS buffer with HOSu as an additive and reactivity boosting agent at a pH of 6.5, 7.0, or 8.0. In the reaction with the MOPS buffer alone, conjugation of the mPEG30k to EPO produced low yields of PEG-EPO conjugates, with the majority of the protein remaining unconjugated at all three pHs tested. As seen in FIG. 6A and detailed in Table 1, only 41% of the EPO formed a conjugate (34% 1:1 conjugate and 7% 2:1 conjugates) at a pH of 8.0. At the lower pH of 7.0, only 16% of the EPO formed a conjugate. At a pH of 6.5, 9% of the EPO formed a conjugate. In contrast, using the MOPS/HOSu buffer, EPO reacted with mPEG30k-NPC efficiently, with a composition favoring the formation of mono- and di-PEGylated-EPO. As seen in FIG. 6B, at pH 8.0, 92% of the EPO formed a conjugate (40% as 1:1, 42% as 1:2, and 10% as 1:3 conjugates). Conjugation proceeded to a higher degree even at the lower pH, 83% conjugation at 7.0 and 62% at 6.5.

As further illustrated in Example 2, boosting the conjugation of mPEG-NPC at lower pH ranges may be especially beneficial for proteins that are not stable at higher pH ranges. Addition of the activating agent is effective to increase at least one of the rate of reaction, the extent of reaction, and/or the time to completion for the reaction. This data further illustrates that PEG-NPC in the absence of the reactivity boosting agent, HOSu, is rather inert at pH 7.

As also detailed in Example 2, conjugates of mPEG30k-EPO were prepared by reacting mPEG30k-SC with EPO in MOPS buffer at a pH of 6.5, 7.0, or 8.0 for comparison. Conjugation of the mPEG30k-SC to EPO reacted efficiently at all pH, however formation of di- and tri-PEGylated-EPO was more highly favored than found using conjugation of mPEG-NPC to EPO in the presence of HOSu at all pHs. As detailed in Table 2, at pH 8.0, 98% of the EPO formed a conjugate (24% as 1:1, 50% as 1:2, and 24% as 1:3 conjugates). At a pH 7.0, 96% of the EPO was conjugated with 29% as 1:1 conjugates, 48% as 2:1 conjugates, and 19% as 3:1 conjugates. At pH 6.5, 90% of the EPO was conjugated with 39% as 1:1 conjugates, 42% as 2:1 conjugates, and 10% as 3:1 conjugates.

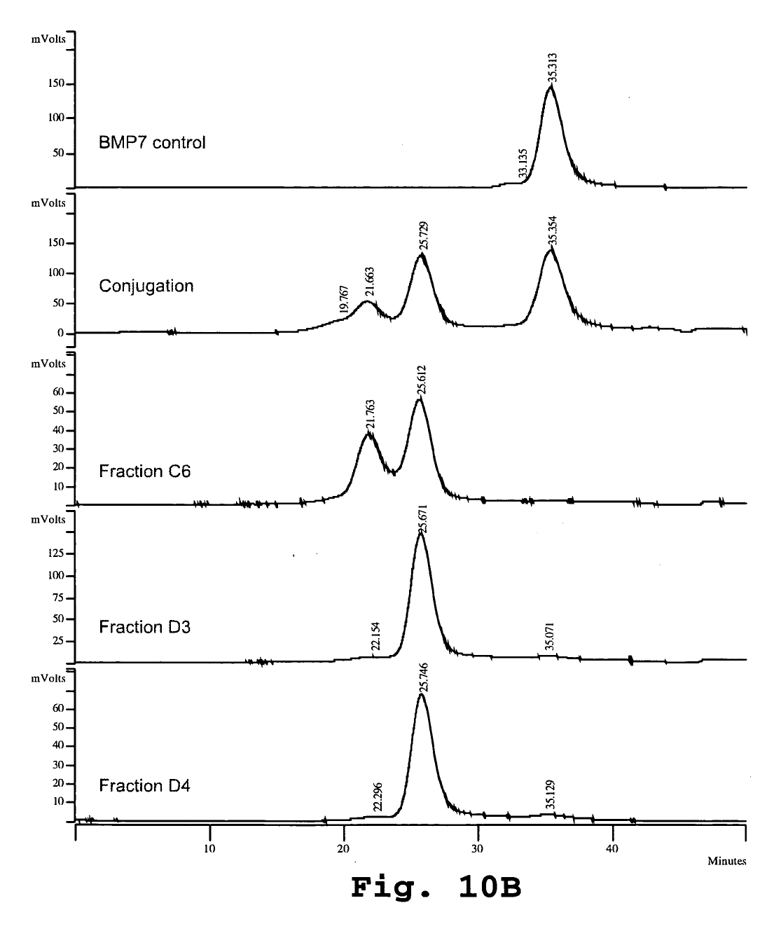

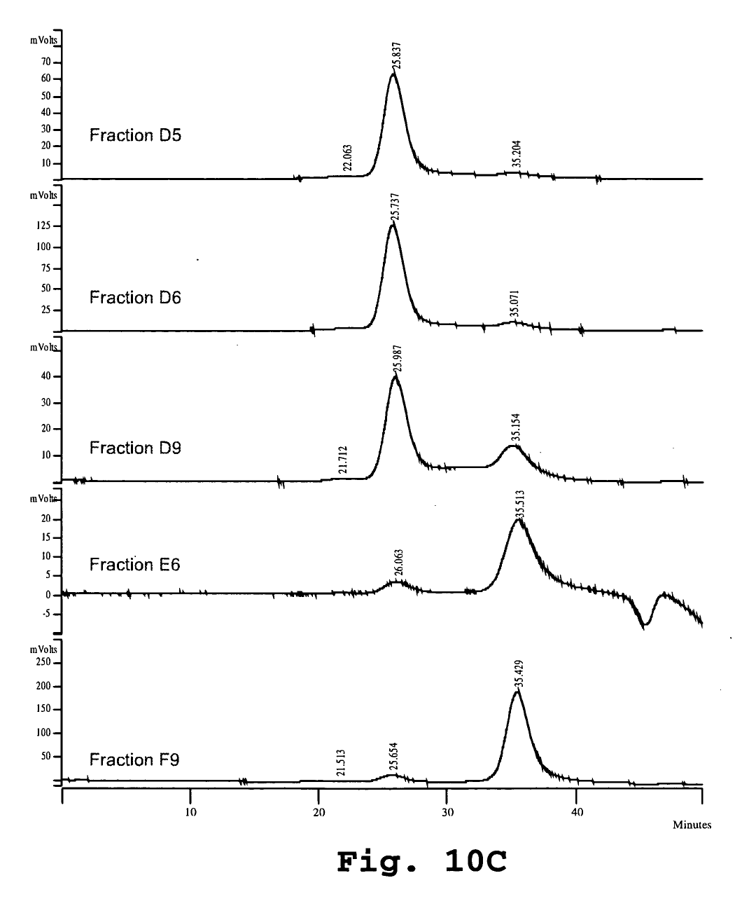

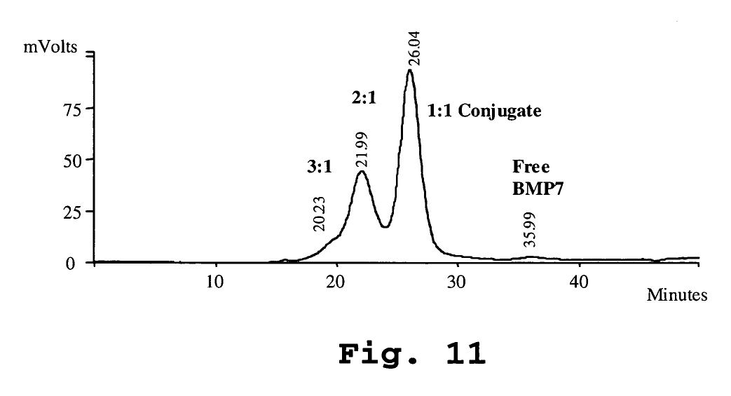

Bone Morphogenic Protein 7 or Bone Morphogenetic Protein 7 (BMP7) is a member of a large, structurally-related subgroup of the TGF-β super family of proteins. BMP7 is not soluble at a pH>7, particularly in the presence of salts. Therefore, any PEGylation reaction must be carried out below pH 7, preferably between pH 6 and 7. As described in Example 5, conjugates of mPEG30k-BMP7 were prepared with HOSu as an activating and buffering agent. The BMP7 reacted with mPEG30k-NPC efficiently, resulting in a composition favoring the formation of mono- and di-PEGylated-BMP7. FIG. 9 shows a trace of the HPLC-SEC analysis of the PEG30k-BMP7 conjugates (top) formed in Example 5 as compared to free BMP7 (bottom). Free BMP7 eluted at about 35 minutes, while the conjugates eluted at about 26 minutes for the 1:1 conjugate, at about 22 minutes for the 2:1 conjugate, and at about 20 minutes for the 3:1 conjugate. The peak area for each of the conjugates and the free BMP7 was calculated and is shown in the figure. 58% of the BMP7 formed a conjugate (42% 1:1 conjugates, 16% 2:1 conjugates, and 4% 3:1 conjugates) at a pH of 6.0. As seen in FIGS. 10A-10C, the ion exchange purification was able to separate unreacted BMP7 and PEG from the PEGylated-BMP7. The conjugates were separated by cation exchange chromatography with the results shown in FIGS. 10A-10C. FIGS. 10B-10C show the results of HPLC-SEC analysis of the C6, D3, D4-D6, D9, E6, and F9 fractions as compared to the BMP7 control and the total conjugation. The majority of the fractions show significant 1:1 (eluted at about 25-26 minutes) and 2:1 (elution at about 22 minutes) conjugation. The purified sample was diluted and analyzed with HPLC-SEC with the results shown in FIG. 11. Free BMP7 eluted at about 36 minutes, where the conjugates eluted at about 25-26 minutes for the 1:1 conjugate, at about 22 minutes for the 2:1 conjugate, and at about 20 minutes for the 3:1 conjugate. The peak area for each of the conjugates and the free BMP7 was calculated with 61% 1:1 PEG-BMP7, 33% 2:1 PEG-BMP7, 5% 3:1 PEG-BMP7, and 1% free BMP7. It should be noted that even higher yield may be obtained by adjusting the concentration of the protein. However, it may not be feasible to modify the concentration of BMP7 at a higher pH as the protein tends to precipitate at higher pH.

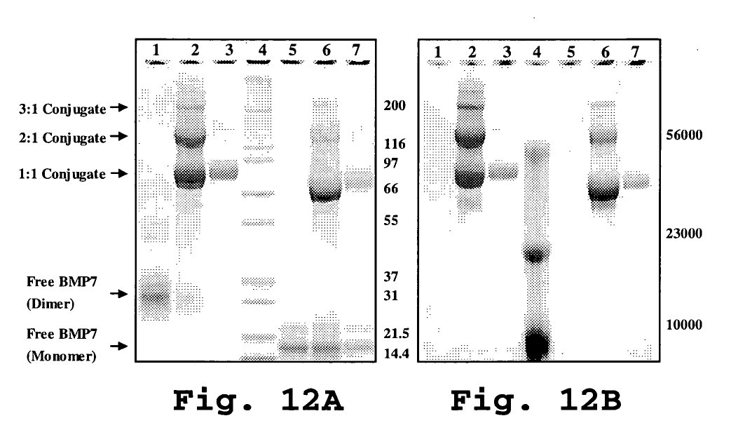

Electrophoresis gels were run with the mPEG30k-BMP7 sample as shown in FIGS. 12A-12B. FIG. 12A shows a gel using a Coomassie blue stain for detection of protein. FIG. 12B shows a gel using an iodine stain for detection of the PEG. For each of the gels, Lane 1 is the BMP7 control, Lane 2 is the purified preparation of PEG30k-BMP7, lane 3 is the PEG30k-BMP7 1:1 conjugate. Lane 4 is a molecular weight (MW) marker for proteins in FIG. 12A and a molecular weight marker for PEG in FIG. 12B. Lane 5 is a BMP7 control reduced, lane 6 is PEG30k-BMP7 reduced, and lane 7 is PEG30k-BMP7 1:1 conjugate reduced. As seen from these results, the majority of the conjugates in the preparation are 1:1 conjugates (see the bands in lane at about 80 kDa). Lane 2 also shows significant 2:1 conjugates by the band at about 116 kDa. Thus, in the case of a relatively insoluble protein like BMP7, the ability to perform an efficient PEGylation reaction at pH 6 allows for preparation of PEG-BMP7 with relatively low species heterogenicity (n=1 to 3).

Lysozyme is an enzyme found in egg whites, milk, tears, and other secretions. It acts as an antibiotic by breaking down the polysaccharide walls of many kinds of bacteria. As described in Example 6, conjugates of mPEG30k-lysozyme were prepared in either a MOPS buffer solution or a MOPS/HOSu buffer solution at a pH of 6.5, 7.0, or 8.0 for comparison. As seen in Table 4, at a pH 8.0, 41% of the lysozyme reacted with mPEG30k-NPC to form a conjugate (34% as 1:1 conjugate and 7% as 2:1 conjugate) in the MOPS buffer. At pH 7.0, only 10% of the lysozyme reacted with mPEG30k-NPC as 1:1 conjugate. At pH 6.5, only 6% of the lysozyme reacted with mPEG30k-NPC as 1:1 conjugate. In the MOPS/HOSu buffer, the lysozyme reacted with the mPEG 30k-NPC to form primarily mono- and di-PEGylated-lysozyme. At pH 8.0, 46% of the lysozyme reacted with mPEG30k-NPC to form a conjugate (38% as 1:1 conjugate, 8% as 2:1 conjugate, and 1% as 3:1 conjugate). At pH 7.0, 40% of the lysozyme reacted with mPEG30k-NPC (34% as 1:1 conjugate and 6% as 2:1 conjugate). At pH 6.5, 30% of the lysozyme reacted with mPEG30k-NPC (27% as 1:1 conjugate and 3% as 2:1 conjugate).

In a preferred embodiment, the total conjugation in the presence of the activating agent is 1 to 7 fold greater than conjugation without the activating agent. In a more preferred embodiment, conjugation with the activating agent is 2 to 5 fold greater than conjugation without the activating agent.

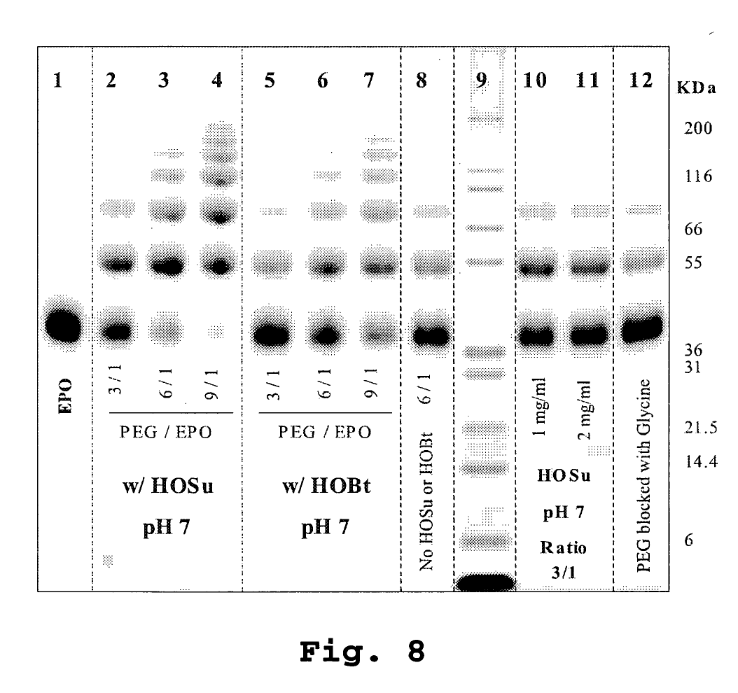

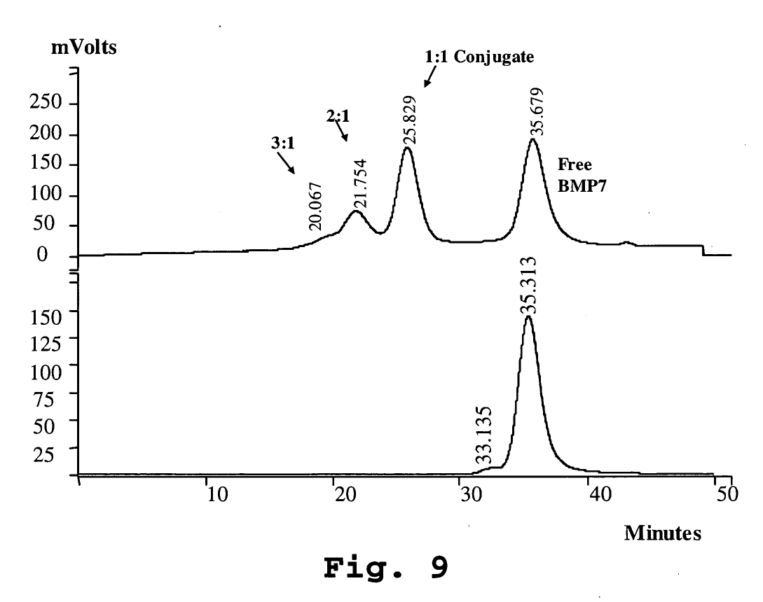

As detailed in Example 3, the rate of hydrolysis of mPEG30k-NPC was measured in MOPS buffer, with and without the presence of HOSu at pH 6.5, 7.0, or 8.0. Briefly, mPEG30k-NPC was added to vials containing either MOPS buffer or MOPS buffer with HOSu. The rate of formation of p-nitrophenol was measured at 400 nm. FIGS. 7A-7B show the rate of formation of p-nitrophenol from mPEG-NPC by hydrolysis at different pH in either a MOPS buffer (FIG. 7A) or in a MOPS buffer in the presence of HOSu (FIG. 7B). For each of FIGS. 7A-7B, hydrolysis at pH 6.5 is represented by ♦, hydrolysis at pH 7.0 is represented by ▪, and hydrolysis at pH 8.0 is represented by ▴. As seen in FIG. 7A, the mPEG30k-NPC in a MOPS buffer hydrolyzed slowly at all pH used (0.0002 OD/min for pH 6.5, 0.0004 OD/min for pH 7.0, and 0.0017 OD/min for pH 8.0), and was fairly stable at pH≦7. However, as seen in FIG. 7B, mPEG30k-NPC was more susceptible to hydrolysis in the presence of an activating agent such as HOSu. The rate of reaction in the presence of the activating agent was 0.021 OD/min at pH 6.5, 0.059 OD/min at pH 7.0, and 0.278 OD/min at pH 8.0. As shown in FIGS. 7A-7B, when HOSu is present in the buffer, the release of para-nitrophenol is dramatically accelerated in an apparent transesterification reaction. Since HOSu has good water solubility, it can be added to buffers at relatively high concentration, which facilitates the transesterification process. By itself, HOSu has a pKa≈6 and is useful as a buffer component at pH 5-7.

The activating agent produces transesterification of the carbonate ester of the mPEG-NPC, which is then hydrolyzed to produce the p-nitrophenol, mPEG-OH, and CO₂. In a preferred embodiment, addition of an activating agent results in at least a 100 fold increase in the rate of reaction. In other embodiments, use of an activating agent results in a 10, 100, or 150 or more fold increase in the rate of reactions.

It will be appreciated that each or any of the temperature, pH and time involved can be varied as needed to maximize yield and/or minimize time for the reaction.

In one embodiment, the method results in formation of moderately PEGylated proteins, and is particularly advantageous for the preparations of PEG-protein comprising 1:1 PEGylated, 2:1 PEGylated, and/or 3:1 PEGylated proteins. Preferably, the reaction produces primarily 1:1 PEGylated protein. In a preferred embodiment, the method produces about 40-45% 1:1 PEGylated protein. In a more preferred embodiment, the method produces about 55-65% 1:1 PEGylated protein. In yet another embodiment, the method produces about 60% 1:1 PEGylated protein. In another embodiment, the method results in a population having about equal molar ratio of 1:1 PEGylated protein to 2:1 PEGylated protein. In other embodiments, the population comprises about 15-40% 1:1 PEGylated protein, about 30-50% 2:1 PEGylated protein, and about 15-40% 3:1 PEGylated protein. The resulting population may further include unmodified protein (nonPEGylated). In preferred embodiments, nonPEGylated protein is present in an amount less than about 20%, 10%, or 5% of the total protein. In another preferred embodiment, nonPEGylated protein is present in an amount of less than about 1% or less than about 0.1% of the total protein. In a preferred embodiment, at least 60-90% of the protein is PEGylated with the present method.

It will be appreciated that the composition of the resulting population may be analyzed according to any suitable method known in the art. In one embodiment, the composition of the population is analyzed by HPLC. Size exclusion chromatography is useful and readily separates the various PEGylated species from each other and from the nonPEGylated protein.

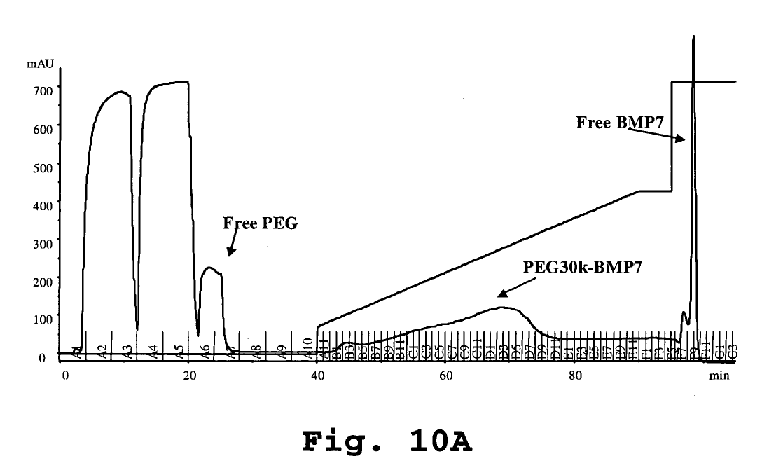

Another variable that can be utilized to maximize yield of PEGylated protein is the ratio of protein to mPEG-NPC and/or the activating agent. As described in Example 4, 0.2, 0.4, and 0.6 mM solutions of mPEG12k-DTB-NPC were used to prepare PEGylated EPO in the presence of HOSu or HOBt. The solutions had a molar ratio of 3, 6, or 9 PEG/EPO and a molar ratio of 100, 50, or 25 HOSu/NPC. FIG. 8 shows the results of the gel electrophoresis. As seen in lanes 2, 3, and 4, increasing the ratio of PEG/EPO results in a greater percentage of PEGylation for the protein indicated by the bands between 55 and 200 kDa. The n=1 pegylated species is represented by the band at 55 kDa, the n=2 pegylated species is represented by the band at about 90 kDa, etc. This trend is also represented by lanes 5, 6, and 7 for the HOBt buffer. In the absence of buffer, very little acylation of EPO occurred, see lane 8. Lanes 10 and 11 show the effect of varying the amount of EPO in the reaction at a PEG/EPO ratio of 3/1. Lane 12 shows the results of using PEG blocked with glycine for 20 minutes and then reacting with EPO for comparison. As seen from the results, both HOSu and HOBt boosted the reactivity of PEG-NPC compared to the buffer alone. It will be appreciated that the starting concentration of protein can further be scaled up or down, as needed, using methods known to those in the art. It should be noted that having a conjugate PEG-protein mixture in vivo assures that the clearance of PEG-protein species is considerably prolonged as conjugates with higher n are cleared slower than those with lower n values. In one embodiment, the starting concentration of protein is between about 0.2 to about 10 mg/ml. In preferred embodiments, the starting concentration of protein is about 1-5 mg/ml.

In another embodiment, the process described herein was used to attach a hydrophilic polymer poly(ethylene glycol) to a structure referred to in the art as a MIMETIBODY™ (see PCT Publication Nos. WO 04/002417; WO 04/002424; WO 05/081687; and WO 05/032460, all of which are incorporated by reference herein). A mimetibody can comprise at least one CH3 region directly linked with at least one CH2 region directly linked with at least one hinge region or fragment thereof directly linked with an optional linker sequence, directly linked to at least one therapeutic peptide, optionally further directly linked with at least a portion of at least one variable antibody sequence. In a preferred embodiment, the mimetibody comprises a pair of a CH3-CH2-hinge-linker-therapeutic peptide fusion polypeptides, the pair linked by association or covalent linkage, such as, but not limited to, a Cys-Cys disulfide bond. For example, an EPO mimetic CH1 deleted mimetibody mimics an antibody structure with its inherent properties and functions, while providing a therapeutic peptide and its inherent or acquired in vitro, in vivo or in situ properties or activities.

Mimetibodies provide at least one suitable property as compared to known proteins, such as, but not limited to, at least one of increased half-life, increased activity, more specific activity, a selected or more suitable subset of activities, less immunogenicity, increased quality or duration of at least one desired therapeutic effect, less side effects, and the like.

Human mimetibodies that are specific for at least one protein ligand or receptor thereof can be designed against an appropriate ligand, such as isolated and/or EPO protein receptor or ligand, or a portion thereof (including synthetic molecules, such as synthetic peptides). Preparation of such mimetibodies are performed using known techniques to identify and characterize ligand binding regions or sequences of at least one protein or portion thereof.

A mimetibody referred to in the art as CNTO528 was selected as a model biomolecule (mimetibody) for PEGylation according to the process described herein. CNTO528 is an Epo receptor agonist, described in PCT Publication WO 04/002417, incorporated by reference herein. Examples 7 and 8 describe reaction of CNTO528 and PEG according to the reaction method described herein.

It will be appreciated that the methods described herein are useful for PEGylating a variety of peptides and proteins that are not soluble and/or not stable at a pH higher than about 8.0 or about 7.0.

In another embodiment, at least one PEG is attached at one or more sites on the protein molecule. It will further be appreciated that conjugation of PEG at more than one site on the protein molecule may increase circulation time.

It will be appreciated that the rate of reactivity is highly dependent on the pH of the buffer in the PEGylation reaction and the accessibility of the individual primary amino groups.

The reaction may be stopped by mixing a free amino compound in the reaction medium. Such a free amino compound includes, but is not limited to, TRIS, lysine, glycine, or any amino with at least one free amino group. In a preferred embodiment, the free amino compound is glycine. In this embodiment, the glycine is preferably used at a concentration of between about 10-100 mM, about 1-50 mM, or about 50-100 mM.

The method may further include a purification step according to known methods in the art. In other embodiments, either the product of the combining step or the product of a purifying step can be concentrated. In one embodiment, the concentrating step can be performed using ultrafiltration or concentration with, for example, a nominal molecular weight limit (NMWL) cutoff filter. Filters for performing such concentrations are commercially available. Occasionally, optimized reactions may not require purification steps to remove excess PEG reagent. However, one of skill in the art will appreciate both purification and concentration steps can be selected among techniques known in the art.

Various patents and publications are cited throughout the specification. Each of these patents or publications is expressly incorporated by reference herein, in its entirety.

### EXAMPLES

The following examples are provided for the purpose of illustrating various presently preferred embodiments of the invention. As such, they are intended to exemplify and clarify, but not to limit the understanding or description of the invention in its several aspects.

Materials and Methods

Erythropoietin (EPO) was obtained from Johnson & Johnson Pharmaceutical Research & Development (Raritan, N.J.), at a protein concentration of 3.56 mg/ml in 20 mM citrate, 100 mM NaCl, pH 6.9 buffer.

Recombinant human Bone Morphogenetic Protein-7 (BMP7) was obtained from CURIS (Cambridge, Mass.), as a lyophilized powder and kept at −70° C.

Nitrophenyl carbonate derivatized methoxy-polyethylene glycol 30,000 Daltons (mPEG30k-NPC) was purchased from NOF Corporation (Tokyo, Japan, #M35525).

Succinimidyl carbonate derivatized methoxy-polyethylene glycol 30,000 Da. (mPEG30k-SC) was prepared by reacting mPEG30k-NPC with N-hydroxysuccinimide in presence of di-isopropyl ethylamine, and purified by crystallization.

N-hydroxysuccinimide (HOSu), 1-hydroxybenzotriazole (HOBt), sodium phosphate (NaPO₄), and 3-[N-Morpholino]propanesulfonic acid sodium salt (MOPS) were purchased from Sigma-Aldrich (St. Louis, Mo.).

### Example 1

### PEGylation of EPO

A. Conjugation

A 10 mM stock solution of mPEG30k-NPC was prepared in acetonitrile. The conjugation buffer was prepared as a stock solution of 100 mM MOPS (3-[N-Morpholino]propanesulfonic acid sodium salt) and 100 mM HOSu (N-hydroxysuccinimide), and the pH was adjusted to 7.0±0.1 with 5 N NaOH. The reaction was initiated by first mixing 5.2 ml of EPO to 2.25 ml of MOPS/HOSu buffer, and 1.194 ml of distilled water. Afterward, 0.356 ml of mPEG30k-NPC was added drop by drop to the mixture, while gently vortexing. The reaction was allowed to proceed for 4 hours at room temperature (21-22° C.) on a rocking mixer, followed by an additional 18 hours at 4° C. The final reaction volume was 9 ml, containing 2 mg/ml (0.066 mM) of EPO, and 0.4 mM of mPEG30k-NPC, giving a molar ratio of 6 PEG/EPO, and 4% acetonitrile. The final HOSu concentration was 25 mM, which is approximately 62 molar excess over mPEG30k-NPC.

B. Analysis by HPLC Size Exclusion Column

The outcome of the conjugation reaction was analyzed by HPLC-SEC using Superose-6 10/300 GL, 1×30 cm column (Amersham Biosciences, Piscataway, N.J.), and 50 mM NaPO₄, 150 mM NaCl, pH 6.5, mobile phase. The sample was diluted 1/20 in the mobile phase, and 50 μl were injected to the column. The flow rate was set at 0.5 ml/min, and elution from the column was monitored by a fluorescence detector set at an excitation wavelength of 295 nm, and emission wavelength of 360 nm (bandwidth 15 nm).

FIG. 2 shows HPLC-SEC chromatograms of the conjugates at the end of the reaction (top) and the parent EPO (bottom). As seen in FIG. 2, the conjugation reaction resulted in the formation primarily of mono- and di-PEGylated species, 50% and 28% respectively.

C. Purification

The conjugation reaction sample was purified by ion exchange preceded by dialysis.

1. Dialysis:

10 mM Tris buffer pH 7.5, using SPECTRA/POR 1 membrane tubing (Spectrum Medical Industries Inc., Los Angeles, Calif.), having a molecular weight cut-off of 6000-8000. The dialysis was carried out at 4° C. The first three buffer exchanges were each performed in 1 liter Tris buffer for 2 hours, the 4th exchange was performed overnight in 3 L Tris buffer, and the final exchange was done in 1 L buffer for 2 hours. At the end of dialysis, the sample was filtered through a 0.45 μm Acrodisc HT Tuffryn membrane syringe filter (PALL Life Sciences, Ann Arbor, Mich.).

2. Separation by Ion Exchange Chromatography:

A quaternary amine anion exchanger column, Source 15Q 4.6×100 mm (Amersham Biosciences, Piscataway, N.J.), 1.7 ml total volume, was equilibrated with 20 column volumes of 10 mM Tris pH 7.5 buffer. Next, 7.8 ml of the dialyzed conjugation sample were loaded on the column. Elution was performed by step gradient using mobile phase A containing 10 mM Tris pH 7.5, and mobile phase B containing 500 mM NaCl in 10 mM Tris pH 7.5, at a flow rate of 1.7 ml/min. The unbound material was washed from the column with 5 column volumes of mobile phase A. The elution started by increasing mobile phase B to 15% (75 mM NaCl) for 10 minutes, then to 20% B (100 mM NaCl) for 15 minutes, and finally to 70% B (350 mM NaCl) for 5 minutes. Fractions were collected at 0.85 ml/fraction through the entire separation. FIG. 3A shows the results of the ion exchange separation. EPO (pl. 4.5 to 5.5) bound to the quarternary amine matrix at pH 7.5 and eluted at a higher counter ion concentration, whereas PEG-EPO interacted with the matrix to a lesser extent, possibly due to the shielding of its negatively charged residue by the PEG chains. The free PEG did not bind to the column.

D. Analysis by HPLC-SEC

To identify the content of the fractions collected throughout the ion exchange separation, aliquots from the fractions were analyzed by HPLC-SEC using TSKgel Super SW3000, 0.46×30 cm column (TOSOH Biosciences LLC, Montgomeryville, Pa.), 50 mM NaPO₄, 150 mM NaCl, pH 6.5, mobile phase, and a flow rate of 0.25 ml/min.

FIGS. 3B-3D shows the HPLC-SEC analysis for the fractions collected through the ion exchange separation in Section C, above. As seen in FIGS. 3B-3D, fractions C3 through D11 (elution time 12.5 to 22.4 min.) contained PEGylated EPO having a majority of 1:1 PEG/EPO conjugate. Those fractions were pooled together in a total volume of 18 ml.

E. Dialysis and Concentration

The pooled sample from the ion exchange separation was dialyzed in 20 mM sodium citrate, 100 mM sodium chloride, pH 6.9 buffer, using SPECTRA/POR 1 tubing described above. The dialysis was carried out at 4° C. The first buffer exchange was done in 2 L citrate/NaCl buffer for 2 days, the 2nd exchange was performed overnight in 2 L Tris buffer, and the final exchange was done in 1 L for 4 hours. The dialyzed sample was then concentrated, under nitrogen at 20 psi, in a 10 ml Amicon ultrafiltration stirred cell (Millipore Corp., Billerica, Mass.), using an OMEGA ultrafiltration membrane disc filter (PALL Life Sciences, Ann Arbor, Mich.), having a molecular weight cut-off of 3000. The sample volume was reduced from 18 ml to 4 ml final volume.

F. Sterile Filtration

The concentrated sample was sterile filtered through 0.22 μm Acrodisc HT Tuffryn membrane syringe filter, and sterilely filled into autoclaved glass vials. All vials were stored at 4° C.

The concentration of the mPEG30k-EPO preparations was determined to be 1.2 mg/ml based on the intrinsic fluorescence of EPO protein and calibrated with the parent EPO.

G. HPLC-SEC Analysis

The purified mPEG30k-EPO sample was analyzed by size exclusion chromatography using Superose-6 10/300 GL column (Amersham Biosciences, Piscataway, N.J.), and 50 mM NaPO4, 150 mM NaCl, pH 6.5, mobile phase. The sample was diluted 1/20 in the mobile phase, and 50 μl were injected to the column. The flow rate was set to 0.5 ml/min, and elution from the column was monitored by a fluorescence detector set at an excitation wavelength of 295 nm, and emission wavelength of 360 nm (bandwidth 15 nm). The results are shown in FIG. 4, where the purified PEGylated-EPO contained 91% and 6% of mono-PEG and di-PEG conjugates respectively. A small amount of unconjugated EPO (4%) was also detected.

H. SDS-PAGE Analysis

The mPEG30k-EPO sample was analyzed by gel electrophoresis under denaturing conditions, using NuPAGE® Bis-Tris 4-12% gradient gel and MOPS-SDS running buffer (Invitrogen Life Technology, Carlsbad, Calif.). Samples and controls were loaded on the gel at 10 μl/well containing 1.5 to 5 μg of protein. The gel was run at a constant voltage of 200 volts for 55 minutes. The gel was first stained in iodine for PEG detection, and subsequently in Coomassie Blue for protein detection. The electrophoresis gels are shown in FIGS. 5A-5B. FIG. 5A shows the iodine stained gel and FIG. 5B shows the Coomassie Blue stained gel. Lane 1 corresponds to a PEG molecular marker, lane 2 corresponds to a PEG30k control, lane 3 corresponds to an EPO control, lane 4 corresponds to the mPEG30k-EPO sample before purification, lane 5 corresponds to the purified mPEG30k-EPO, and lane 6 corresponds to a protein molecular weight marker.

The composition of the purified mPEG30k-EPO sample determined by SDS-PAGE confirmed the HPLC-SEC results of FIGS. 5A-5B. The sample contained mono and di-PEGylated EPO only. However, the intensity of the bands, in both gels, was not representative of the actual percentage of each conjugate species, due to the presence of PEG on the protein. Coomassie Blue appeared to stain PEG as well as EPO, yet, at a much lower specificity then iodine stain (FIG. 5A). A higher amount of PEG per protein in the conjugate induced a higher band intensity than the protein itself would have showed.

### Example 2

### Conjugation of EPO to mPEG30k-NPC With and Without N-Hydroxysuccinimide

Three MOPS buffer solutions were prepared at 100 mM, and the pH values were adjusted to 6.5, 7.0, and 8.0 respectively, with 6 N HCl.

Three MOPS/HOSu buffer solutions were prepared at 100 mM MOPS/100 mM HOSu, and the pHs were adjusted to 6.5, 7.0, and 8.0 respectively, with 5 N NaOH.

A 10 mM (302.6 mg/ml) stock solution of mPEG30k-NPC was made in acetonitrile. A 10 mM (302 mg/ml) stock solution of mPEG30k-SC was made in acetonitrile. A stock solution of glycine was made with 500 mM in 10 mM Tris pH 7.5 buffer.

A total of 9 conjugation reactions were assembled. In the first set, EPO was reacted with mPEG30k-NPC in MOPS buffer at pH, 6.5, 7.0, or 8.0. In the second set, EPO was reacted with mPEG30k-NPC in MOPS/HOSu buffer at pH, 6.5, 7.0, or 8.0. In the third set, EPO was reacted with mPEG30k-SC in MOPS buffer at pH 6.5, 7.0, or 8.0. All the reactions were carried out at room temperature (21-22° C.) for 4 hours, while mixing, then transferred to 4° C. for an additional 18 hours.

The final concentrations in the reaction vials were 2 mg/ml (0.066 mM) for EPO, and 0.4 mM for mPEG30k, giving a molar ratio of 6 PEG/EPO. The final acetonitrile amount was 4%. In the reactions containing MOPS/HOSu buffer, the final HOSu concentration was 25 mM, which is equivalent to 62 molar excess over mPEG.

At the end of the incubation, glycine was added to all the reactions, to a final concentration of 25 mM, and allowed to react for 20 minutes at room temperature.

Samples from the conjugation reactions were diluted 1:25 in 50 mM NaPO₄, 150 mM NaCl, pH 6.5 buffer, and 50 μl were injected to Superose-6 10/300 GL column (Amersham Biosciences, Piscataway, N.J.) to determine the amount of conjugation of the PEG and EPO. The mobile phase was 50 mM NaPO₄, 150 mM NaCl, pH 6.5, run at 0.5 ml/min flow rate. A fluorescence detector was connected to the column outlet, and set to an excitation wavelength of 295 nm, and emission wavelength of 360 nm (bandwidth 15 nm). The chromatograms were analyzed by Star Chromatography Workstation 6.2 (Varian Inc., Walnut Creek, Calif.) and the results were expressed in percent of peak areas as shown in Table 1. The results for the HPLC-SEC analysis of the mPEG30k-EPO conjugates in the MOPS buffer alone are shown in FIG. 6A and the results for mPEG30k-EPO conjugates in the MOPS/HOSu buffer are shown in FIG. 6B.

TABLE 1
Percent areas of EPO and its conjugates formed by EPO reaction with
mPEG30k-NPC

| | MOPS Buffer | | | MOPS/HOSu Buffer | | |
|---|---|---|---|---|---|---|
| | pH 6.5 | pH 7.0 | pH 8.0 | pH 6.5 | pH 7.0 | pH 8.0 |
| Unconjugated | 91% | 84% | 59% | 38% | 17% | 8% |
| EPO | | | | | | |
| 1:1 Conjugate | 9% | 15% | 34% | 45% | 45% | 40% |
| 2:1 Conjugate | 0% | 1% | 7% | 16% | 32% | 42% |
| 3:1 Conjugate | 0% | 0% | 0% | 1% | 6% | 10% |

TABLE 2
Percent areas of EPO and its conjugates formed by
EPO reaction with mPEG30k-SC

| | MOPS Buffer | | |
|---|---|---|---|
| | pH 6.5 | pH 7.0 | pH 8.0 |
| Unconjugated EPO | 10% | 4% | 2% |
| 1:1 Conjugate | 39% | 29% | 24% |
| 2:1 Conjugate | 42% | 48% | 50% |
| 3:1 Conjugate | 10% | 19% | 24% |

### Example 3

### Hydrolysis of mPEG30k-NPC in the Presence of N-Hydroxysuccinimide

mPEG30k-NPC solution, MOPS buffer, MOPS/HOSu buffer were prepared as described in Example 2.

Forty microliters of the 10 mM mPEG30k-NPC were added to 3 vials, each containing 960 μl of 100 mM MOPS buffer solution at a pH of 6.5, 7.0, and 8.0, respectively. The mixture from each vial was immediately transferred to a 96-well microtiter plate in triplicates (300 μl/well). The plate optical density (OD) reading started at 400 nm and was collected for 2 hours in 2 minute reading intervals using a Vis range A400. Similar assays were repeated with the MOPS/HOSu buffer. The results are shown in FIGS. 7A-7B.

### Example 4

### PEGylation with HOSu and HOBt at various PEG/EPO Ratios

A. Reactions with HOSu in Phosphate Buffer

EPO was reacted at 2 mg/ml to 0.2, 0.4, and 0.6 mM mPEG12k-DTB-NPC, in conjugation buffer containing 100 mM sodium phosphate and 20 mM HOSu, at pH 7. The reaction was allowed to proceed for 5 hours at room temperature (22-24° C.) on a rocking mixer. During the reactions, the PEG/protein molar ratios were 3/1, 6/1, or 9/1, and HOSu/NPC molar ratios were 100/1, 50/1, and 25/1. At the end of incubation, the reactions were stopped with 9 mM glycine.

The samples were analyzed by SDS-PAGE and the results are displayed as lanes 2, 3, and 4 in FIG. 8.

B. Reactions with HOBt in Phosphate Buffer

Reactions were conducted as in section A, above except that the conjugation buffer was prepared as a stock solution of 100 mM sodium phosphate and 20 mM HOBt at pH 7. The samples were analyzed by SDS-PAGE as in section A, above, and the results are displayed as lanes 5, 6, and 7 in FIG. 8.

C. Reactions in Phosphate Buffer

EPO was reacted at 2 mg/ml to mPEG12k-DTB-NPC at 0.4 mM, in conjugation buffer containing 100 mM sodium phosphate, at pH 7. The reaction was allowed to proceed for 5 hours at room temperature (22-24° C.) on a rocking mixer. During the reactions, the PEG/protein molar ratio was 6/1. At the end of incubation, the reactions were stopped with 9 mM glycine.

The samples were analyzed by gel electrophoresis as in section A, above. The results are displayed as lane 8 in FIG. 8.

D. Reactions with HOSu in Phosphate Buffer at Various EPO Concentrations

EPO was reacted at 1 or 2 mg/ml to mPEG12k-DTB-NPC (at 0.1 or 0.2 mM), in conjugation buffer containing 100 mM sodium phosphate and 5 or 10 mM HOSu, at pH 7. The reaction was allowed to proceed for 5 hours at room temperature (22-24° C.) on a rocking mixer. During the reactions, the PEG/protein molar ratio was 3/1, and HOSu/NPC molar ratio was 50/1. At the end of incubation, the reactions were stopped with 9 mM glycine.

The samples were analyzed by gel electrophoresis as in section A, above, the results of which are displayed in lanes 10 and 11 in FIG. 8.

### Example 5

### PEGylation of BMP7

A. Conjugation

A 1.4 mg/ml BMP7 stock solution was prepared in 25 mM HOSu (N-hydroxysuccinimide), pH 6. A 10 mM stock solution of mPEG30k-NPC was prepared in acetonitrile. A 12.86 ml of BMP7 (18 mg) was mixed with 4.24 ml of HOSu buffer, pH 6. Afterward, 0.9 ml of mPEG30k-NPC were added drop by drop to the mixture, while gently vortexing. The reaction was incubated for 16 hours at room temperature (21-22° C.) on a rocking mixer. The final reaction volume was 18 ml containing 1 mg/ml (0.028 mM) of BMP7, 0.5 mM of mPEG30k-NPC, 5% acetonitrile, and a molar ratio of 18 PEG/BMP7. The final HOSu concentration was 24 mM, which is approximately 48 molar excess over mPEG30k-NPC. The reaction was quenched with 10 mM glycine for 1 hour at room temperature.

B. Analysis by HPLC Size Exclusion Column

The outcome of the conjugation reaction was analyzed by HPLC-SEC using Superose-6 10/300 GL, 1×30 cm column (GE Healthcare, Piscataway, N.J.), and 25 mM Tris, 300 mM NaCl, 6 M Urea, pH 6.5, mobile phase. The sample was diluted 1/20 in the mobile phase, and 50 μl were injected to the column. The flow rate was set at 0.5 ml/min, and elution off the column was monitored by a fluorescence detector set at an excitation wavelength of 295 nm, and emission wavelength of 360 nm (bandwidth 15 nm) with the results shown in FIG. 9. Approximately 42% of the protein remained unconjugated, and 58% of the BMP7 was PEGylated producing a majority of mono-PEGylated protein, 38%.

C. Purification

1. Dialysis: 10 mM Na Acetate buffer pH 5, using SPECTRA/POR 1 membrane tubing (Spectrum Medical Industries Inc., Los Angeles, Calif.), having a molecular weight cut-off of 6000-8000. The dialysis was carried out at 4° C. At the end of dialysis, the sample was filtered through a 0.45 μm Acrodisc HT Tuffryn membrane syringe filter (PALL Life Sciences, Ann Arbor, Mich.).

2. Separation by Ion Exchange Chromatography:

A sulphopropyl cation exchanger column, Source 15S PE 4.6×100 mm (GE Healthcare, Piscataway, N.J.), 1.7 ml total volume, was equilibrated with 20 column volumes of 10 mM sodium acetate pH 5 buffer. Next, 20 ml of the dialyzed conjugation sample were loaded on the column. Elution was performed by gradient elution using mobile phase A containing 10 mM sodium acetate pH 5, mobile phase B1 containing 1 M NaCl in 10 mM sodium acetate pH 5, and mobile phase B2 containing 6 M urea, 1 M NaCl, 10 mM sodium acetate pH 5, at a flow rate of 1 ml/min. The unbound material to the column was washed out with 40 ml of mobile phase A. The gradient elution started by increasing mobile phase B1 from 10% to 60% in 50 minutes, then to 100% B2 (1 M NaCl, 6 M urea) for 10 minutes at 2 ml/min. Fractions were collected at 1 ml/fraction throughout the elution step with the results shown in FIGS. 10A-10C. Unreacted PEG did not bind to the column and came out with flow-through material. PEGylated BMP7 started eluting at approximately 150 mM NaCl and was completed at approximately 400 mM NaCl. As seen in FIG. 10A, free BMP7 was eluted at the end with 1 M NaCl containing 6 M urea.

D. Analysis by HPLC-SEC

In order to identify the content of the fractions collected throughout the ion exchange separation, aliquots from the fractions were analyzed by HPLC-SEC using Superose-6 10/300 GL column described above and the results are depicted in FIGS. 10B-10C.

E. Dialysis and Concentration

Fractions B2 to D3, from the ion exchange separation, containing the PEGylated protein were pooled concentrated and dialyzed in 20 mM sodium acetate, 5% mannitol, pH 4.5 buffer, under nitrogen at 20 psi, in a 10 ml Amicon ultrafiltration stirred cell (Millipore Corp., Billerica, Mass.), using an OMEGA ultrafiltration membrane disc filter (PALL Life Sciences, Ann Arbor, Mich.), having a molecular weight cut-off of 3000. The sample volume was brought down to approximately 3.5 ml final volume.

F. Sterile Filtration

The concentrated sample was sterile filtered through 0.22 μm Acrodisc HT Tuffryn membrane syringe filter, and sterilely filled into autoclaved glass vials. All vials were stored at 4° C. Approximately a total of 1.2 mg of PEG30k-BMP7 were obtained from the purification, as determined by the protein assay described below.

G. Protein Determination Assay:

The protein determination assay was based on the fluorescent characteristic of the protein intrinsic tryptophan. BMP7 was used as a standard, and serial dilutions were made at 6.25, 12.5, 25, 50, 100, and 200 μg/ml in 20 mM sodium acetate, 5% mannitol, pH 4.5 buffer. The mPEG30k-BMP7 sample was diluted 1:10 and 1:20 in the same buffer. The standards and test samples were transferred to a black microtiter plate, at 200 μl/well, in triplicates with the results shown in Table 3 and FIG. 11. The plate was read in a fluorometer set at an excitation wavelength of 295 nm (2 nm slit), and emission wavelength of 360 nm (10 nm slit).

TABLE 3
Determination of protein concentration in PEG30k-BMP7 purified
sample, by the intrinsic tryptophan fluorescence assay, using BMP7
as standard.

| | | Estimated | | | |
|---|---|---|---|---|---|
| | | BMP7 | | | |
| | | concentra- | | Estimated | |
| | | tion | | concentration × | |
| Test | Mean | in test | Dilu- | dilution | Protein |
| sample | Intensity | sample | tion | factor | concentration |
| dilution | cps | μg/ml | factor | mg/ml | mg/ml |
| 1/20 | 25521 | 16.79 | 20 | 0.34 | 0.35 |
| 1/10 | 51879 | 35.45 | 10 | 0.35 | |

H. HPLC-SEC Analysis

The purified mPEG30k-BMP7 sample was analyzed by size exclusion chromatography using a Superose-6 10/300 GL column described above. The sample was diluted to 50 μg/ml in the mobile phase, and 50 μl were injected to the column. The flow rate was set to 0.5 ml/min, and elution off the column was monitored by a fluorescence detector set at an excitation wavelength of 295 nm, and emission wavelength of 360 nm (bandwidth 15 nm) with the results shown in FIG. 11.

I. SDS-PAGE Analysis

The mPEG30k-BMP7 sample was analyzed by gel electrophoresis under denaturing conditions, using NuPAGE® Bis-Tris 4-12% gradient gel and MOPS-SDS running buffer (Invitrogen Life Technology, Carlsbad, Calif.). Samples and controls were loaded on 2 gels at 10 μl/well containing 1.5 to 5 μg of protein. The gels were run at a constant voltage of 200 volts for 55 minutes. One gel was stained in iodine for PEG detection (FIG. 12A), and the other in Coomassie Blue (FIG. 12B) for protein detection. The resulting gels are shown in FIGS. 12A-12B.

### Example 6

### PEGylation of Lysozyme

A 10 mM stock solution of mPEG30k-NPC was prepared in acetonitrile. The lysozyme was prepared at a stock solution of 4.16 mg/ml in 20 mM sodium citrate buffer at pH 6.9, containing 100 mM NaCl. Three MOPS buffer solutions were prepared at 100 mM, and the pH was adjusted to 6.5, 7.0, and 8.0 respectively, with 6 N HCl. Three MOPS/HOSu buffer solutions were prepared at 100 mM MOPS/100 mM HOSu, and the pH was adjusted to 6.5, 7.0, and 8.0 respectively, with 6 N NaOH. A stock solution of glycine was prepared with 500 mM glycine in 10 mM TRIS pH 7.5 buffer.

A total of 6 conjugation reactions were assembled. In the first set, lysozyme was reacted to mPEG30k-NPC in MOPS buffer at pH 6.5, 7.0, or 8.0. In the second set, lysozyme was reacted to mPEG30k-NPC in MOPS/HOSu buffer at pH 6.5, 7.0, or 8.0. All of the reactions were carried out at room temperature (21-22° C.) for 4 hours, while mixing, then transferred to 4° C. for an additional 18 hours.

The final concentrations in the reaction vials were 2 mg/ml (0.14 mM) for lysozyme and 0.4 mM for mPEG30k, resulting in a molar ratio of 3/1 PEG/lysozyme. The final acetonitrile amount was 4%. In the reactions including MOPS/HOSu buffer, the final HOSu concentration was 25 mM, which is equivalent to 62 molar excess over mPEG-NPC.

Samples were analyzed with a Superose-6 10/300 GL column (Amersham Biosciences, Piscataway, N.J.) and fluorescence detector essentially as described in Example 2 to determine the amount of conjugation of the PEG and lysozyme. The results are described in Table 4 below.

TABLE 4
Lysozyme conjugation to mPEG30k-NPC with and without HOSu at
various pH

| | MOPS Buffer | | | MOPS/HOSu Buffer | | |
|---|---|---|---|---|---|---|
| | pH 6.5 | pH 7.0 | pH 8.0 | pH 6.5 | pH 7.0 | pH 8.0 |
| Unconjugated | 94% | 90% | 59% | 70% | 60% | 54% |
| lysozyme | | | | | | |
| 1:1 Conjugate | 6% | 10% | 34% | 27% | 34% | 38% |
| 2:1 Conjugate | 0% | 0% | 7% | 3% | 6% | 8% |
| 3:1 Conjugate | 0% | 0% | 0% | 0% | 0% | 1% |

### Example 7

### Preparation of PEG30kCNTO528

CNTO528, an Epo receptor agonist as described in PCT Publication No. WO 04/002417, was selected as a model biomolecule (mimetibody). In CNTO528, the sequence of an Epo mimetic peptide (EMP-1) known to require dimerization for bioactivity is fused to the hinge and Fc portion of IgG1, resulting in an active Epo receptor agonist. There are 21 lysine residues in the Fc and hinge portion of CNTO528 and the Epo mimetic peptide has one lysine residue. In addition, there are two amino terminal groups on a single mimetibody molecule (46 total potential sites). Although the Fc portion contributes to a longer circulation time compared to the free peptide, even longer circulation may be desired for improved dosing regimens.

Amine-directed PEGylation of CNTO528 was performed as follows, and according to the reaction scheme described above. A 10 mM solution of PEG30k-NPC in acetonitrile was prepared just prior to use. The mimetibody CNTO528 (Lot# FV2413A) was prepared as described in PCT Publication Nos. WO 04/002417; WO 04/002424; WO 05/081687; and WO 05/032460. A buffer of 100 mM HEPES and 100 mM N-hydroxysuccinimide (HOSu), pH 7.5 was prepared.

PEG30k-NPC was used in 10-fold molar excess to CNTO528. PEG solution was added to CNTO528 in buffer and water to a final protein concentration of 4 mg/mL, a final buffer concentration of 25 mM HEPES/HOSu, and a final PEG concentration of 0.645 mM. The reaction was allowed to proceed at room temperature (21-22° C.) in the dark on a rocking mixer for 4 hours and then placed in 4° C. overnight. The reaction was stopped with 30 mM final concentration of glycine.

The crude reaction material was dialyzed in 10 mM citrate, pH 5.0, and then purified by cation exchange chromatography (SP HP 1 mL or 5 mL column, Amersham Biosciences) using a NaCl elution gradient. The reaction material was characterized by size-exclusion chromatography (SEC) using Superose-6 (Amersham Biosciences) in a 50 mM sodium phosphate and 100 mM NaCl, pH 7.4 mobile phase. Following analysis by SEC, fractions were pooled to obtain the desired species ratio. The SEC chromatogram details are summarized in Table 5 below.

TABLE 5
Chromatogram Details for Crude Reaction Material

| Peak | Ret Time | % Peak | |
|---|---|---|---|
| No | (min) | Area | PEG:MMB |
| 1 | 20.75 | 55.2 | 3:1 |
| 2 | 22.8 | 31.1 | 2:1 |
| 3 | 27.25 | 12.5 | 1:1 |
| 4 | 37.76 | 1.2 | free MMB |

Next, the pooled conjugate mixture was dialyzed into phosphate buffered saline (PBS), pH 7.2, and filtered using a 0.2 μm pore-size membrane to sterilize. PEG30K-CNTO 528 was placed in a sterile glass vial at 2 mL±0.1 mL, as determined by A280 (1.0 mg/mL). This conjugate mixture was characterized by SEC as above and the results are shown in Table 6 below.

TABLE 6
Chromatogram Details for Purified Reaction Material

| Peak No | Ret Time (min) | Peak Area | % Peak Area | PEG:MMB |
|---|---|---|---|---|
| 1 | 21.99 | 1497829 | 30.0 | 3:1 |
| 2 | 23.26 | 3455284 | 48.2 | 2:1 |
| 3 | 27.49 | 2448511 | 21.4 | 1:1 |
| 4 | 38.25 | 78898 | 0.5 | free MMB |

### Example 8

### Preparation of PEG30kCNTO528

Conjugate was prepared as described in Example 7, with the following changes to the amounts of the reaction components. Final concentrations for CNTO528, HEPES/HOSu buffer, and PEG30k-NPC were 5.6 mg/mL, 35 mM, and 0.9 mM respectively. The reaction was stopped with 40 mM final concentration of glycine.

The conjugate was characterized by size-exclusion chromatography (SEC) using Superose-6 (Amersham Biosciences) in a 50 mM sodium phosphate and 150 mM NaCl, pH 7.0 mobile phase and the results from the chromatograms for crude and purified materials are shown in the Tables 7 and 8 below.

TABLE 7
Chromatogram Details for Crude Reaction Material

| | Ret Time | % Peak | |
|---|---|---|---|
| Peak No | (min) | Area | PEG:MMB |
| 1 | 21.62 | 60.3 | 3:1 |
| 2 | 23.82 | 29 | 2:1 |
| 3 | 28.42 | 10.4 | 1:1 |
| 4 | 39.8 | 0.3 | free MMB |

TABLE 8
Chromatogram Details for Purified Reaction Material

| | Ret Time | % Peak | |
|---|---|---|---|
| Peak No | (min) | Area | PEG:MMB |
| 1 | 22.51 | 20.0 | 3:1 |
| 2 | 24.03 | 46.2 | 2:1 |
| 3 | 28.23 | 32.7 | 1:1 |
| 4 | 39.58 | 1.1 | free MMB |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method of optimizing 1:1 PEG-protein in a heterogeneous population of PEG-protein molecules comprising:
combining a PEG derivatized acylating agent and a protein in the presence of an activating agent at a pH of less than about 7.0 or neutral pH.

2. The method according to claim 1, wherein said PEG derivatized acylating agent is mPEG-NPC.

3. The method according to claim 1, wherein said activating agent is selected from the group consisting of HOSu, HOBt, and HOAt.

4. The method according to claim 1, wherein said protein is selected from the group consisting of EPO, BMP7, lysozyme, and a mimetibody.

5. A method of PEGylating a protein that is insoluble or unstable at a pH higher than about 7.0 or neutral pH, comprising:
reacting the protein with a PEG derivatized acylating agent and an activating agent at a pH lower than about 7.0.

6. The method according to claim 5, wherein said PEG derivatized acylating agent is mPEG-NPC.

7. The method according to claim 5, wherein said activating agent is selected from the group consisting of HOSu, HOBt, and HOAt.

8. The method according to claim 5, wherein said protein is selected from the group consisting of EPO, BMP7, lysozyme, and a mimetibody.

9. A method to PEGylate a protein to form predominately 1:1 PEG-protein at a pH of less than about 8 and greater than the pKa of the additive to boost the rate of the reaction, comprising:
reacting the protein with a PEG derivatized acylating agent and an activating agent at a pH lower than about 8.0.

10. The method according to claim 9, wherein said PEG derivatized acylating agent is mPEG-NPC.

11. The method according to claim 9, wherein said activating agent is selected from the group consisting of HOSu, HOBt, and HOAt.

12. The method according to claim 9, wherein said protein is selected from the group consisting of EPO, BMP7, lysozyme, and a mimetibody.
